(19) United States
(12) Patent Application Publication
(10) Pub. No.: US 2025/0171816 A1
(43) Pub. Date: May 29, 2025

(54) COMPOSITE ENGINEERED BACTERIA AND METHOD FOR PRODUCING D-PSICOSE

(71) Applicant: HENAN ZHONGDA HENGYUAN BIOTECHNOLOGY STOCK CO., LTD., Luohe (CN)

(72) Inventors: Sanying WANG, Luohe (CN); Ziheng JIN, Luohe (CN); Yanjun WEN, Luohe (CN); Linzheng LI, Luohe (CN); Chenhui WEN, Luohe (CN); Rongrong XIE, Luohe (CN); Tianyi PAN, Luohe (CN)

(21) Appl. No.: 19/041,885

(22) Filed: Jan. 30, 2025

Related U.S. Application Data
(63) Continuation of application No. PCT/CN2024/108982, filed on Jul. 31, 2024.

(30) Foreign Application Priority Data
Jul. 19, 2023 (CN) 202310885687.0

(51) Int. Cl. C12P 19/02; C12N 9/92; C12N 15/75; C12P 19/24; C12R 1/125

(52) U.S. Cl. CPC C12P 19/02; C12N 9/92; C12N 15/75; C12P 19/24; C12R 2001/125; C12Y 501/03

(57) ABSTRACT

The present invention relates to an enzyme combination, genetically engineered bacteria, and application thereof in producing D-psicose. Glucose isomerase and D-psicose 3-epimerase from specific sources are separately co-expressed in Bacillus subtilis, a combination enzyme containing glucose isomerase and D-psicose 3-epimerase is obtained through fermentation, and the combination enzyme is used to catalyze a glucose substrate to perform isomerization to eventually prepare D-psicose. The method provided in the present invention can clearly increase a conversion rate of D-psicose, which greatly reduces the time of converting glucose into D-psicose. In addition, inexpensive glucose is used as a raw material, so that the production costs of D-psicose are greatly reduced. In the present invention, fructose syrup may be synchronously produced, so that the input for a whole set of production lines of fructose syrup is omitted, thereby greatly improving the economic benefits.

This application is a Continuation Application of PCT/CN2024/108982, filed on Jul. 31, 2024, which claims priority to Chinese Patent Application No. 202310885687.0, filed on Jul. 19, 2023, which is incorporated by reference for all purposes as if fully set forth herein.

A Sequence Listing XML file named “10025_0122.xml” created on Jan. 30, 2025 and having a size of 21,419 bytes, is filed concurrently with the specification. The sequence listing contained in the XML file is part of the specification and is herein incorporated by reference in its entirety.

### TECHNICAL FIELD

The present invention relates to the field of bioengineering technologies, and in particular, to composite engineered bacteria and a method for producing D-psicose.

### BACKGROUND

D-psicose is a rare sugar having 70% sweetness of sucrose but low energy, and is an ideal substitute for sucrose. In addition, D-psicose has a lower absorption rate than other sweeteners, so that the absorption of fructose and glucose by an organism can be reduced, and the deposition of fat is reduced, thereby reducing the risks of type 2 diabetes, obesity, and other diseases. At present, there are also researches showing that D-psicose also has the functions of lowering blood lipid and blood sugar. The United States Food and Drug Administration (FDA) has approved D-psicose as generally recognized as safe (GRAS) and can be used as a component of a food additive. D-psicose has broad application prospect in food and health care products. However, fructose is mainly used as a raw material in current existing processes, and raw material costs are high. Glucose is used as a raw material in a few processes. However, this has a long production period, consumes more labor and materials, increases production costs, and therefore is not conducive to industrial production.

### SUMMARY

In view of this, the present invention provides composite engineered bacteria and a method for producing D-psicose. In the present invention, glucose isomerase and D-psicose 3-epimerase from specific sources are used to catalyze glucose to perform isomerization to generate D-psicose. The method can clearly increase a conversion rate of D-psicose, reduce the production time of D-psicose, and reduce production costs.

To achieve the foregoing inventive objective, the following technical solutions are provided in the present invention:

A method for producing D-psicose includes:
(1) separately transferring genes encoding glucose isomerase derived from Thermus thermophilus and D-psicose 3-epimerase derived from Ruminococcus sp. into Bacillus subtilis to obtain engineered bacteria of GI and engineered bacteria of DPE;
(2) mixing and inoculating the engineered bacteria of GI and the engineered bacteria of DPE into a fermentation medium for fermentation, and performing bacterial cell disruption to obtain a crude enzyme preparation containing the glucose isomerase and the D-psicose 3-epimerase; and
(3) catalyzing glucose by using the crude enzyme preparation as a catalyst to perform isomerization to obtain D-psicose.

In the present invention, in Step (1), an amino acid sequence of the glucose isomerase is any one selected from the following:
(a) the sequence shown in SEQ ID NO: 1;
(b) an amino acid sequence that is obtained by substituting, deleting or adding one or more amino acids to/from the sequence shown in SEQ ID NO: 1 and has protein activity remaining unchanged; or
(c) an amino acid sequence that is at least 90% homologous with the sequence shown in SEQ ID NO: 1 and has protein activity same as or similar to that of the sequence shown in SEQ ID NO: 1; and
an amino acid sequence of the D-psicose 3-epimerase is any one selected from the following:
(A) the sequence shown in SEQ ID NO: 2;
(B) an amino acid sequence that is obtained by substituting, deleting or adding one or more amino acids to/from the sequence shown in SEQ ID NO: 2 and has protein activity remaining unchanged; or
(C) an amino acid sequence that is at least 90% homologous with the sequence shown in SEQ ID NO: 2 and has protein activity same as or similar to that of the sequence shown in SEQ ID NO: 2.

In the present invention, in Step (1), the gene encoding the glucose isomerase is optimized according to the codon preference of the Bacillus subtilis, where a nucleotide sequence after the optimization is shown in SEQ ID NO: 3; and
the gene encoding the D-psicose 3-epimerase is optimized according to the codon preference of the Bacillus subtilis, where a nucleotide sequence after the optimization is shown in SEQ ID NO: 4.

In the present invention, the glucose isomerase GI is derived from Thermus thermophilus, and has the registry number being WP_244348257.1 and the amino acid sequence being SEQ ID NO: 1. The D-psicose 3-epimerase DPE is derived from Ruminococcus sp., and has the registry number being MBS6425357.1 and the amino acid sequence being SEQ ID NO: 2.

In the present invention, through long-term research, it is found that rates of conversion of glucose into psicose by catalyzing a glucose substrate by using enzyme combinations from different sources are different, to eventually obtain an optimal combination with a clearly higher conversion rate than other combinations, namely, a combination of glucose isomerase derived from Thermus thermophilus and D-psicose 3-epimerase derived from Ruminococcus sp. Specifically, the amino acid sequence of the glucose isomerase is SEQ ID NO: 1, and the amino acid sequence of the D-psicose 3-epimerase is SEQ ID NO: 2.

In the present invention, the fermentation in Step (2) is specifically cultivation at 37° C. and 200 rpm for 48 h.

In the present invention, in Step (2), the fermentation medium is 10 g of peptone, 5 g of yeast extract, 2.5 g of monopotassium phosphate, 15 g of potassium hydrogen phosphate, 0.1 g of manganese chloride tetrahydrate, 0.1 g of magnesium sulfate heptahydrate, and 6 g of glucose, and water is added to fix the volume to 1 L.

In the present invention, before the engineered bacteria are inoculated into the fermentation medium for mixed fermentation, Step (2) further includes the step of preparing a seed broth with the engineered bacteria: inoculating the engineered bacteria into an LB seed culture medium, and performing overnight cultivation at 37° C. and 200 rpm to obtain the seed broth.

In the present invention, the bacterial cell disruption in Step (2) is specifically: mixing lysozyme and a fermentation broth according to a proportion of a mass volume ratio being 0.1‰ (i.e., a final concentration of lysozyme is 0.1 g/L), and performing reactions at 37° C. and 200 rpm for 0.5 h to 4 h. In some specific embodiments, the condition of performing the bacterial cell disruption is performing reactions at 37° C. and 200 rpm for 1 h.

In the present invention, a temperature of the isomerization in Step (3) is 60° C., and a time ranges from 2 h to 24 h. In some specific embodiments, the temperature of the isomerization is 60° C., and a time is 2 h, 4 h, 6 h, 12 h, or 24 h.

In the present invention, after the isomerization, the method further includes sequentially filtering, purifying, chromatographically separating, and concentrating reaction products to separately obtain the D-psicose and fructose. For the filtration, a ceramic membrane with a pore size ranging from 8 nm to 10 nm and a nanofiltration membrane ranging from 100 daltons to 300 daltons are sequentially used for filtration. The purification includes using cation resin and anion resin sequentially to remove anions and cations. In specific embodiments, the model of the cation resin is D001-FD, and the model of the anion resin is D354-FD;

The present invention further provides composite engineered bacteria, including Bacillus subtilis expressing glucose isomerase derived from Thermus thermophilus and Bacillus subtilis expressing D-psicose 3-epimerase derived from Ruminococcus sp. In some specific embodiments, the Bacillus subtilis is Bacillus subtilis WB600.

In the present invention, a method for constructing the engineered bacteria is as follows:
(1) A protein sequence (GI, SEQ ID NO: 1) of glucose isomerase derived from Thermus thermophilus and a protein sequence (DPE, SEQ ID NO: 2) of the D-psicose 3-epimerase derived from Ruminococcus sp. are selected, where after codon optimization is performed on encoded genes thereof and whole gene synthesis is performed by GenScript Biotech, the sequence of the optimized gene of GI is SEQ ID NO: 3, the sequence of the optimized gene of DPE is SEQ ID NO: 4, and the genes are connected to a vector pWB980 through homologous recombination to obtain recombinant plasmids pWB980-GI and pWB980-DPE. Gene segments of SacB signal peptides on the plasmids are removed during recombination. The genes of GI and DPE are controlled to be constitutive intracellular expression through the p43 promoter.
(2) The recombinant plasmids pWB980-GI and pWB980-DPE are respectively transformed into the Bacillus subtilis WB600 to obtain recombinant Bacillus subtilis WB600/GI and WB600/DPE.

The present invention further provides application of the composite engineered bacteria in producing D-psicose, or application thereof in preparing blood sugar and lipid lowering products.

In specific embodiments of the present invention, the preparation method of D-psicose includes the following steps:
(1) inoculating the foregoing two engineered bacteria (WB600/GI and WB600/DPE) into an LB seed culture medium respectively, and performing overnight cultivation at 37° C. and 200 rpm for enrichment, to obtain a seed broth with an OD600 value ranging from 3 to 6;
(2) jointly inoculating seed broths of two recombinant strains obtained in Step (1) into the same fermentation medium according to a proportion of 0.1% (v/v) for mixed fermentation, and performing cultivation at a fermentation temperature of 37° C. and 200 rpm for 48 h to obtain a fermentation broth;
(3) adding lysozyme to the fermentation broth obtained in Step (2) according to 0.1‰ (w/v), and performing reactions at 37° C. and 200 rpm for 1 h for bacterial cell disruption to release endoenzyme to obtain a crude enzyme preparation;
(4) adding glucose according to a final concentration of 600 g/L to the crude enzyme preparation obtained in Step (3), and performing reactions at 60° C. for 24 h to obtain invert syrup;
(5) performing filtration and impurity removal by using a ceramic membrane with a pore size ranging from 8 nm to 10 nm on the invert syrup obtained in Step (4) with a flux of 50 L/m2/h to obtain a filtrate;
(6) performing filtration, impurity removal, and decoloration by using a nanofiltration membrane with a molecular weight cut-off ranging from 100 daltons to 300 daltons on the filtrate obtained in Step (5) with a flux of 10 L/m²/h to obtain a filtrate;
(7) passing the filtrate obtained in Step (6) through cation resin and anion resin sequentially to remove anions and cations, where the model of the cation resin is D001-FD, and the model of the anion resin is D354-FD, to obtain a desalted sugar solution;
(8) chromatographically separating the desalted sugar solution in Step (7) to obtain an AD solution being a psicose sugar solution and a BD solution being a mixed sugar solution of glucose and fructose, where it is controlled in the process of the chromatographic separation that the concentration of the sugar solution ranges from 15% to 25%, the temperature ranges from 50° C. to 60° C., the pressure ranges from 0.2 MPa to 0.3 MPa, the water-material mass ratio is 1:2, and the throughput is 2.2 kg/kg/d; and
(9) concentrating the AD solution and the BD solution obtained in Step (8) to a solid content of 75% to obtain D-psicose syrup and fructose syrup.

In the present invention, glucose isomerase and D-psicose 3-epimerase from specific sources are separately co-expressed in Bacillus subtilis, a combination enzyme containing glucose isomerase and D-psicose 3-epimerase is obtained through fermentation, and the combination enzyme is used to catalyze a glucose substrate to perform isomerization to eventually prepare D-psicose. The method provided in the present invention can clearly increase a conversion rate of D-psicose, which greatly reduces the time of converting glucose into D-psicose. In addition, in the present invention, inexpensive glucose is used as a raw material, so that the production costs of D-psicose are greatly reduced. In the present invention, fructose syrup may be synchronously produced, so that the input for a whole set of production lines of fructose syrup is omitted, thereby greatly improving the economic benefits. Compared with the prior art, the present invention has the following beneficial effects:
(1) In the present invention, glucose is converted into D-psicose within a short time through catalysis by using an enzyme from a specific source, which clearly reduces the production time, greatly improves the economic benefits, and is conducive to industrial production.
(2) In the present invention, inexpensive raw material glucose is used as a substrate, so that the production costs of D-psicose are greatly reduced.
(3) In the present invention, fructose syrup may be synchronously produced while D-psicose is produced, so that the input for a whole set of production lines of fructose syrup can be omitted.

### BRIEF DESCRIPTION OF THE DRAWINGS

FIG. 1 is maps of recombinant plasmids pWB980-GI and pWB980-DPE;

FIG. 2 is an electrophoresis image of SDS-PAGE, where tracks 1 to 4 respectively represent: 1: WB600/pWB980, 2: WB600/pWB980-GI, 3: WB600/pWB980-DPE, and 4: WB600/pWB980-GI+WB600/pWB980-DPE; and

FIG. 3, FIG. 4 and FIG. 5 are liquid detection chromatographs.

### DESCRIPTION OF THE EMBODIMENTS

The present invention provides composite engineered bacteria and a method for producing D-psicose. A person skilled in the art may refer to the content herein to appropriately improve process parameters for implementation. It is to be particularly noted that all similar replacements and modifications will be apparent to a person skilled in the art, and are all considered to be included in the present invention. The methods and applications of the present invention have been described by means of preferred embodiments, and it is obvious that a related person can implement and apply the technology of the present invention by making modifications or appropriate changes and combinations to the methods and applications herein without departing from the content, spirit, and scope of the present invention.

Experimental materials used in the present invention are all common commercially available products and can be purchased in the market.

The present invention is further described below with reference to the accompanying drawings.

### Embodiment 1: The Construction and Functional Verification of Engineered Bacteria of Glucose Isomerase (GI) in the Present Invention

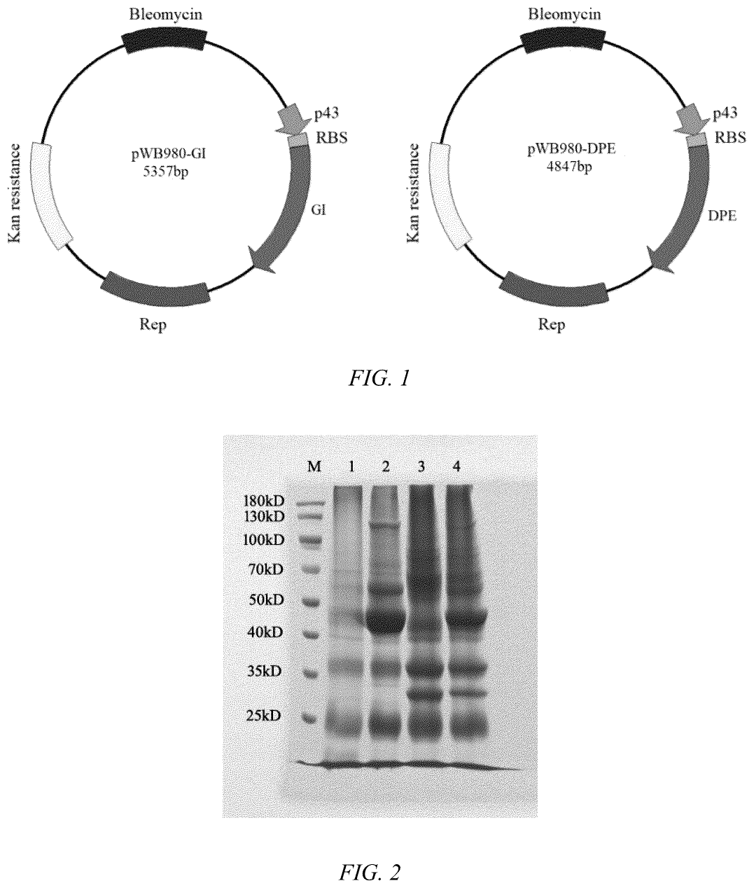

(1) A protein sequence (GI, SEQ ID NO: 1) of glucose isomerase derived from Thermus thermophilus is selected, where encoded genes thereof are optimized according to the codon of Bacillus subtilis, the sequence of the optimized gene of GI is SEQ ID NO: 3, and whole gene synthesis is performed by GenScript Biotech. The synthesized gene is connected to a pUC57 vector and is named pUC57-GI.
(2) PCR amplification is then performed by using the synthesized recombinant plasmid pUC57-GI as a template and GI-F and GI-R as primers, and gel extraction and purification are performed to obtain a GI segment.
(3) PCR amplification is performed by using a plasmid vector pWB980 as a template and pWB980-F1 and pWB980-R1 as primers, and gel extraction and purification are performed to remove a linear gene segment P1 of a SacB signal peptide.
(4) The GI segment and the linear gene segment P1 of pWB980 are connected according to the specification of the homologous recombination kit, and a connection product is electrotransferred to a competent cell of the Bacillus subtilis WB600 to obtain recombinant Bacillus subtilis named WB600/GI. The recombinant plasmid is named pWB980-GI (the map of the plasmid is shown in FIG. 1). Verification is performed through the PCR of a transformant colony and sequencing and analysis (Primer sequences are shown in Table 1).

TABLE 1
Primer sequences of the present invention

| Primer name | Primer sequence 5′-3′ |
|---|---|
| GI-F | ATAAAAAAGGAGACATGAACGATGTATGAACCAAAACC |
| | CGA |
| | (SEQ ID NO: 7) |
| GI-R | TCTTGGAATTGTGCTGAAGCTTTAACCACGCACCCCTA |
| | ACAGATA |
| | (SEQ ID NO: 8) |
| pWB980-F1 | AGCTTCAGCACAATTCCAAGA |
| | (SEQ ID NO: 9) |
| pWB980-R1 | CGTTCATGTCTCCTTTTTTATGTACTG |
| | (SEQ ID NO: 10) |
| DPE-F | ATAAAAAAGGAGACATGAACGATGAAATATGGTATTTA |
| | TTACGCTTATTGGG |
| | (SEQ ID NO: 11) |
| DPE-R | TCTTGGAATTGTGCTGAAGCTTAGACTTTCAAATACAT |
| | GTTTTACAAAGTG |
| | (SEQ ID NO: 12) |
| pWB980-F2 | AGCTTCAGCACAATTCCAAGA |
| | (SEQ ID NO: 13) |
| pWB980-R2 | CGTTCATGTCTCCTTTTTTATGTACTG |
| | (SEQ ID NO: 14) |
| GI-JD-F | ACAGCCATTGAACATACGGT |
| | (SEQ ID NO: 15) |
| GI-JD-R | CCTTGGTAACCGCTAGACT |
| | (SEQ ID NO: 16) |
| DPE-JD-F | ACAGCCATTGAACATACGGT |
| | (SEQ ID NO: 17) |
| DPE-JD-R | TTCCATGCCCATCATAATATCATATT |
| | (SEQ ID NO: 18) |

(5) Positive transformants that appear on an LB culture medium plate containing kanamycin are picked and inoculated into 5 ml of an LB liquid medium containing 50 mg/L of kanamycin for overnight cultivation at 37° C. and 200 rpm; and are inoculated into more than 1 L of a fermentation medium containing 50 mg/L of kanamycin with an inoculum size of 0.1% for cultivation at 37° C. and 200 rpm for 48 h. Lysozyme powder is added according to 0.1‰ to continue to perform reactions at 37° C. and 200 rpm for 1 h, and a GI crude enzyme preparation is obtained (an electrophoresis result of SDS-PAGE is shown in FIG. 2).
(6) Glucose is added according to a final concentration of 600 g/L to the crude enzyme preparation obtained above for reactions at 60° C. for 24 h. 0.02 ml of a reaction solution is taken and diluted 50 times with pure water. Water bathing is performed at 100° C. for 10 min to inactivate enzyme. After centrifugation at 10000 rpm for 10 min, filtration is performed with a 0.22-μm microporous filter membrane. High performance liquid chromatography analysis is performed on the filtrate.

Bacillus subtilis WB600 with empty plasmids pWB980 is used for blank control, and other operation conditions are kept the same.

The high performance liquid chromatography is performed according to the following conditions: an Agilent high performance liquid chromatograph 1200; an analytical column: a Waters Sugar-Pak I chromatographic column; a mobile phase: pure water; a flow rate: 0.3 ml/min, and a column temperature: 80 degrees; and a detector: a differential refractive index detector. The foregoing samples are analyzed by using pure substances of glucose, fructose, and D-psicose produced by the Sigma Corporation as standard substances, and a sample size is 10 μl.

Liquid chromatography analysis results show that the peaking durations of glucose, fructose, and D-psicose are respectively 14.1 min, 17.9 min, and 26.9 min.

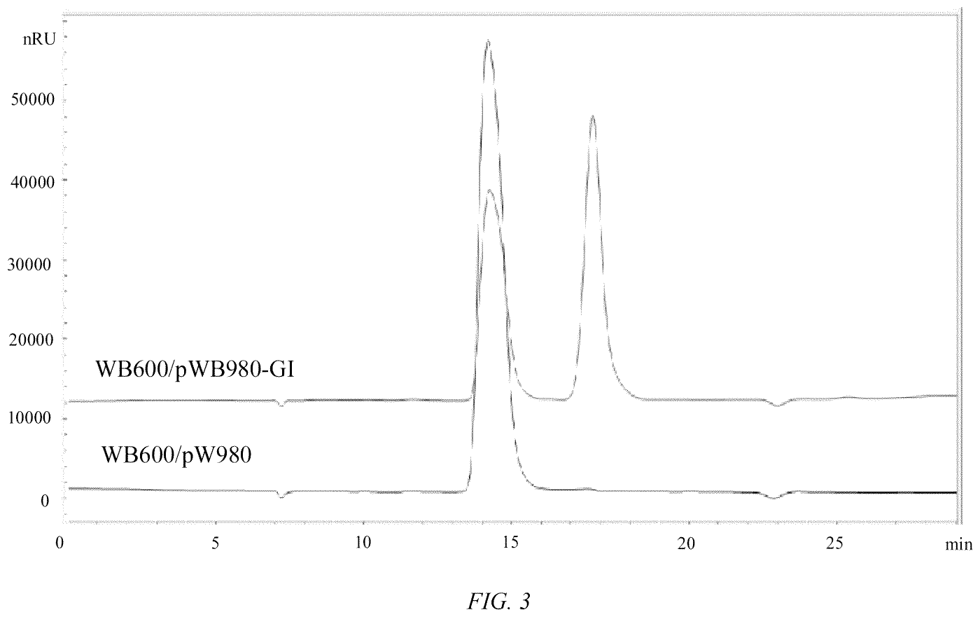

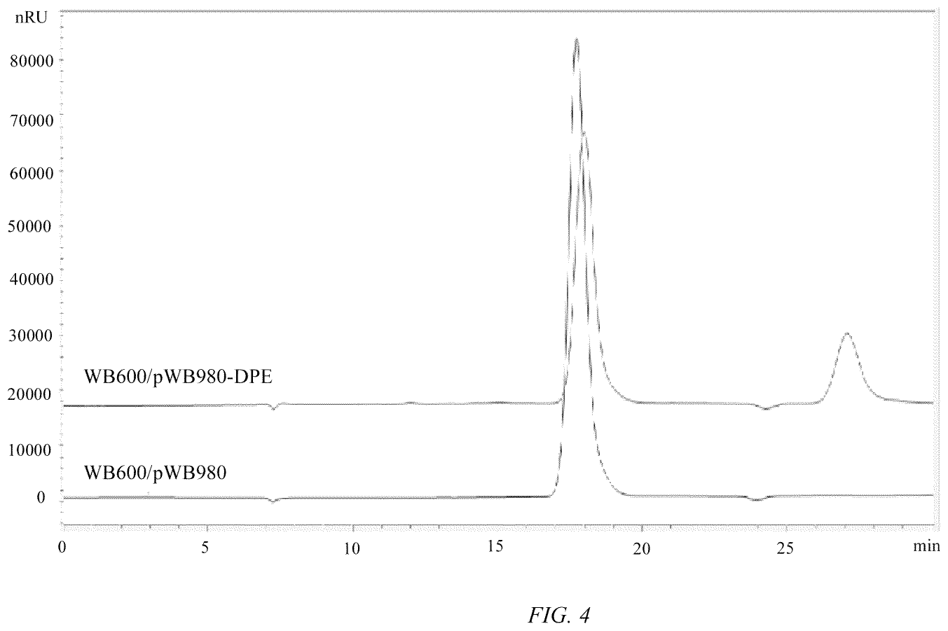

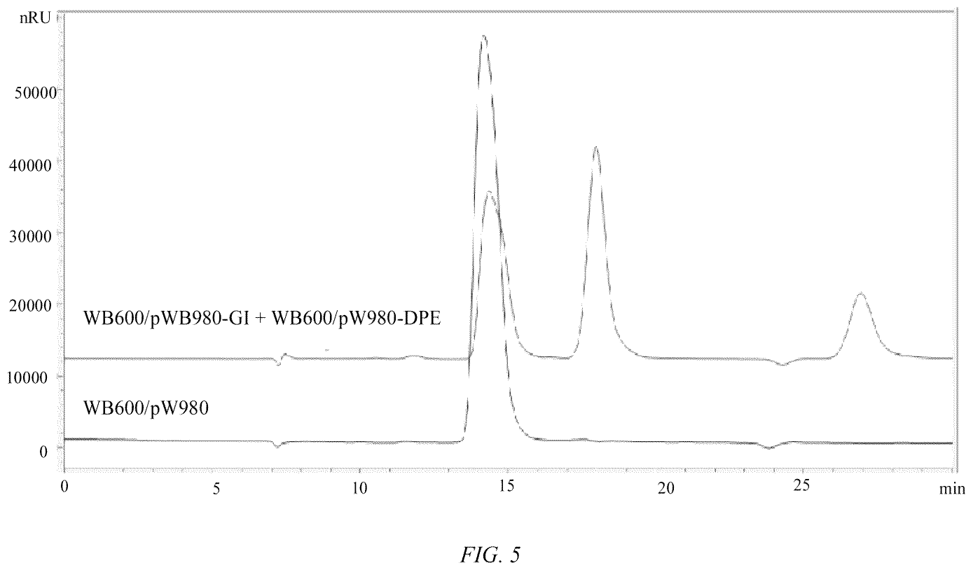

There is only glucose in the liquid chromatograph of the blank control (Bacillus subtilis WB600/pWB980); and there are both glucose and fructose in the liquid chromatograph of the experimental group (Bacillus subtilis WB600/pWB980-GI) (the liquid chromatograph is shown in FIG. 3). The results show that the GI crude enzyme preparation prepared by using the foregoing method can convert glucose into fructose. Through the calculation of peak areas, the converted sugar solution contains 330 g/L of glucose and 270 g/L of fructose, a ratio of which is 55:45.

### Embodiment 2: The Construction and Functional Verification of Engineered Bacteria Expressing D-Psicose 3-Epimerase (DPE)

(1) A protein sequence of D-psicose 3-epimerase (DPE, SEQ ID NO: 2) derived from Ruminococcus sp. is selected, where encoded genes thereof are optimized according to the codon of Bacillus subtilis, the sequence of the optimized gene of DPE is SEQ ID NO: 4, and whole gene synthesis is performed by GenScript Biotech. The synthesized gene is connected to a pUC57 vector and is named pUC57-DPE.
(2) After codon optimization is performed on the encoded genes, whole gene synthesis is performed by GenScript Biotech. The synthesized gene is connected to a pUC57 vector and is named pUC57-DPE.
(3) PCR amplification is then performed by using the synthesized recombinant plasmid pUC57-DPE as a template and DPE-F and DPE-R as primers, and gel extraction and purification are performed to obtain a DPE segment.
(4) PCR amplification is performed by using a plasmid vector pWB980 as a template and pWB980-F2 and pWB980-R2 as primers, and gel extraction and purification are performed to remove a linear gene segment P2 of a SacB signal peptide.
(5) The DPE segment and the linear gene segment P2 of pWB980 are connected according to the specification of the homologous recombination kit, and a connection product is electrotransferred to a competent cell of the Bacillus subtilis WB600 to obtain recombinant Bacillus subtilis named WB600/DPE. The recombinant plasmid is named pWB980-DPE (the map of the plasmid is shown in FIG. 1). Verification is performed through the PCR of a transformant colony and sequencing and analysis (Primer sequences are shown in Table 1).
(6) Positive transformants with correct PCR and sequencing of the colony are picked and inoculated into 5 ml of an LB liquid medium containing 50 mg/L of kanamycin for overnight cultivation at 37° C. and 200 rpm; and are inoculated into more than 1 L of a fermentation medium containing 50 mg/L of kanamycin with an inoculum size of 0.1% for cultivation at 37° C. and 200 rpm for 48 h. Lysozyme is added according to 0.1% o to continue to perform reactions at 37° C. and 200 rpm for 1 h, and a DPE crude enzyme preparation is obtained (an electrophoresis result of SDS-PAGE is shown in FIG. 2).
(7) Fructose is added according to a final concentration of 600 g/L to the crude enzyme preparation obtained above for reactions at 60° C. for 24 h. 0.02 ml of a reaction solution is taken and diluted 50 times with pure water. Water bathing is performed at 100° C. for 10 min to inactivate enzyme. After centrifugation at 10000 rpm for 10 min, filtration is performed with a 0.22-μm microporous filter membrane. High performance liquid chromatography analysis is performed on the filtrate.

Bacillus subtilis WB600 with empty plasmids pWB980 is used for blank control, and other operation conditions are kept the same.

The high performance liquid chromatography is performed according to the following conditions: an Agilent high performance liquid chromatograph 1200; an analytical column: a Waters Sugar-Pak I chromatographic column; a mobile phase: pure water; a flow rate: 0.3 ml/min, and a column temperature: 80° C.; and a detector: a differential refractive index detector. The foregoing samples are analyzed by using pure substances of glucose, fructose, and D-psicose produced by the Sigma Corporation as standard substances, and a sample size is 10 μl.

Liquid chromatography analysis (see FIG. 4) results show that the peaking durations of glucose, fructose, and D-psicose are respectively 14.1 min, 17.9 min, and 26.9 min.

There is only fructose in the liquid chromatograph of the blank control (Bacillus subtilis WB600/pWB980); and there are both fructose and D-psicose in the liquid chromatograph of the experimental group (Bacillus subtilis WB600/pWB980-DPE) (the liquid chromatograph is shown in FIG. 5). The results show that the DPE crude enzyme preparation prepared by using the foregoing method can convert fructose into D-psicose. Through the calculation of peak areas, the converted sugar solution contains 426 g/L of fructose and 174 g/L of D-psicose, a ratio of which is 71:29.

### Embodiment 3: Production of Fructose Syrup and D-Psicose Through Mixed Fermentation of Engineered Bacteria of GI and Engineered Bacteria of DPE

Specific steps are as follows:
(1) inoculating the two engineered bacteria in Embodiments 1 and 2 into an LB seed culture medium respectively, and performing overnight cultivation at 37° C. and 200 rpm for enrichment, to obtain a seed broth with an OD600 value ranging from 3 to 6;
(2) jointly inoculating seed broths of two recombinant strains obtained in Step (1) into the same fermentation medium according to a proportion of 0.1% (v/v) for mixed fermentation, and performing cultivation at a fermentation temperature of 37° C. and 200 rpm for 48 h to obtain a fermentation broth;
(3) adding lysozyme to the fermentation broth obtained in Step (2) according to 0.1‰ (w/v), and performing reactions at 37° C. and 200 rpm for 1 h for bacterial cell disruption to release endoenzyme to obtain a crude enzyme preparation;
(4) adding glucose according to a final concentration of 600 g/L to the crude enzyme preparation obtained in Step (3), and performing reactions at 60° C. for 24 h to obtain invert syrup;
(5) performing filtration and impurity removal by using a ceramic membrane with a pore size ranging from 8 nm to 10 nm on the invert syrup obtained in Step (4) with a flux of 50 L/m2/h to obtain a filtrate;
(6) performing filtration, impurity removal, and decoloration by using a nanofiltration membrane with a molecular weight cut-off ranging from 100 daltons to 300 daltons on the filtrate obtained in Step (5) with a flux of 10 L/m²/h to obtain a filtrate;
(7) passing the filtrate obtained in Step (6) through cation resin and anion resin sequentially to remove anions and cations, where the model of the cation resin is D001-FD, and the model of the anion resin is D354-FD, to obtain a desalted sugar solution;
(8) chromatographically separating the desalted sugar solution in Step (7) to obtain an AD solution being a psicose sugar solution and a BD solution being a mixed sugar solution of glucose and fructose, where it is controlled in the process of the chromatographic separation that the concentration of the sugar solution ranges from 15% to 25%, the temperature ranges from 50° C. to 60° C., the pressure ranges from 0.2 MPa to 0.3 MPa, the water-material mass ratio is 1:2, and the throughput is 2.2 kg/kg/d; and
(9) concentrating the AD solution and the BD solution obtained in Step (8) to a solid content of 75% to obtain D-psicose syrup and fructose syrup.

### Comparative Example 1

Glucose isomerase (GI, refer to SEQ ID NO: 1 in the records of CN113980880 for the nucleic acid sequence) and D-psicose 3-epimerase (DPE, refer to SEQ ID NO: 3 in the records of CN113980880 for the nucleic acid sequence) in Patent CN113980880 are selected. Engineered bacteria of Bacillus subtilis expressing glucose isomerase and engineered bacteria of Bacillus subtilis expressing D-psicose 3-epimerase are respectively constructed according to the methods in Embodiments 1 and 2 of the present invention, and then invert syrup containing glucose, fructose, and psicose is prepared according to the method in Embodiment 3 of the present invention.

### Comparative Example 2 (GI is not Congeneric (is 56% Homologous with the GI in the Present Invention), and DPE is the Same)

Glucose isomerase (GI, refer to SEQ ID NO: 1 in the records of CN113980880 for the nucleic acid sequence) in Patent CN113980880 is selected, and engineered bacteria of Bacillus subtilis expressing GI is constructed according to the method in Embodiment 1 of the present invention. Invert syrup containing glucose, fructose, and psicose is prepared according to the method in Embodiment 3 of the present invention by using the constructed bacteria expressing GI and the constructed bacteria expressing DPE in the present invention.

### Comparative Example 3 (GI is not Congeneric (is 97% Homologous with the GI in the Present Invention), and DPE is the Same)

Glucose isomerase (GI, the registry number is WP_126200404.1) derived from Thermus scotoductus is selected. After codon optimization (the nucleic acid sequence of GI is SEQ ID NO: 5), engineered bacteria of Bacillus subtilis expressing GI is constructed according to the method in Embodiment 1 of the present invention. Invert syrup containing glucose, fructose, and psicose is prepared according to the method in Embodiment 3 of the present invention by using the constructed bacteria expressing GI and the constructed bacteria expressing DPE in the present invention.

### Comparative Example 4 (GI is the Same, and DPE is Different (is 40% Homologous with the DPE in the Present Invention))

D-psicose 3-epimerase (DPE, refer to SEQ ID NO: 3 in the records of CN113980880 for the nucleic acid sequence) in Patent CN113980880 is selected, and engineered bacteria of Bacillus subtilis expressing DPE is constructed according to the method in Embodiment 2 of the present invention. Invert syrup containing glucose, fructose, and psicose is prepared according to the method in Embodiment 3 of the present invention by using the constructed bacteria expressing DPE and the constructed bacteria expressing GI in the present invention.

### Comparative Example 5 (GI is the Same, and DPE is Different (is 69% Homologous with the DPE in the Present Invention))

D-psicose 3-epimerase (DPE, WP_183684385.1) derived from Oribacterium sinus is selected. After codon optimization (the nucleic acid sequence is SEQ ID NO: 6), engineered bacteria of Bacillus subtilis expressing DPE is constructed according to the method in Embodiment 2 of the present invention. Invert syrup containing glucose, fructose, and psicose is prepared according to the method in Embodiment 3 of the present invention by using the constructed bacteria expressing DPE and the constructed bacteria expressing GI in the present invention.

### Experimental Example

Invert syrup is prepared according to the methods in Embodiment 3 and Comparative examples 1 to 5. Samples of reactions for 2 h, 4 h, 6 h, 12 h, and 24 h are respectively taken. HPLC is performed to detect the content of glucose, fructose, and psicose, and the production rates of psicose are calculated. See Table 2 for results.

TABLE 2

| | | | Equilibrium | |
|---|---|---|---|---|
| | | Generated | conversion rate | Production |
| | Reaction | amount (g/L) | (%) of glucose | rate (g/L/h) |
| Group | time (h) | of psicose | and psicose | of psicose |
| Embodiment 1 | 0 | 0 | 17.17 | 0 |
| | 2 | 103 | | 51.5 |
| | 4 | 103 | | 25.75 |
| | 6 | 103 | | 17.17 |
| | 12 | 103 | | 8.58 |
| | 24 | 103 | | 4.29 |
| Comparative | 0 | 0 | 16.33 | 0 |
| example 1 | 2 | 45 | | 22.5 |
| | 4 | 64 | | 16 |
| | 6 | 98 | | 16.33 |
| | 12 | 98 | | 8.17 |
| | 24 | 98 | | 4.08 |
| Comparative | 0 | 0 | 17.33 | 0 |
| example 2 | 2 | 48 | | 24 |
| | 4 | 85 | | 21.25 |
| | 6 | 104 | | 17.33 |
| | 12 | 104 | | 8.67 |
| | 24 | 104 | | 4.33 |
| Comparative | 0 | 0 | 17.83 | 0 |
| example 3 | 2 | 30 | | 15 |
| | 4 | 67 | | 16.75 |
| | 6 | 90 | | 15 |
| | 12 | 107 | | 8.92 |
| | 24 | 107 | | 4.46 |
| Comparative | 0 | 0 | 16.33 | 0 |
| example 4 | 2 | 53 | | 26.5 |
| | 4 | 98 | | 24.5 |
| | 6 | 98 | | 16.33 |
| | 12 | 98 | | 8.17 |
| | 24 | 98 | | 4.08 |
| Comparative | 0 | 0 | 15.00 | 0 |
| example 5 | 2 | 19 | | 9.5 |
| | 4 | 41 | | 10.25 |
| | 6 | 62 | | 10.33 |
| | 12 | 90 | | 7.5 |
| | 24 | 90 | | 3.75 |

As can be learned from the foregoing results, although the efficiency of producing psicose by catalyzing glucose with an enzyme in production of engineered strains in Comparative examples 1 to 5 and the present invention are close, all ranging from 15% to 18 %, the durations taken to reach the equilibrium conversion rate are clearly different, being respectively 6 h, 6 h, 12 h, 4 h, 12 h, and 2 h. The enzyme combination from a specific source in the present invention takes the shortest time, and is clearly lower than those in the other comparative examples; and in addition, has the largest production rate of psicose, and is clearly higher than those of the other comparative examples. Therefore, it may be determined that the combination of glucose isomerase derived from Thermus thermophilus and D-psicose 3-epimerase derived from Ruminococcus sp. provided in the present invention has the highest catalytic activity for glucose, and is the optimal combination.

The foregoing descriptions are preferred implementation manners of the present invention. It should be noted that for a person of ordinary skill in the art, several improvements and modifications may further be made without departing from the principle of the present invention. These improvements and modifications should also be deemed as falling within the protection scope of the present invention.

What is claimed is:

1. A method for producing D-psicose, comprising:
(1) separately transferring genes encoding glucose isomerase derived from Thermus thermophilus and D-psicose 3-epimerase derived from Ruminococcus sp. into Bacillus subtilis to obtain engineered bacteria of GI and engineered bacteria of DPE;
(2) mixing and inoculating the engineered bacteria of GI and the engineered bacteria of DPE into a fermentation medium for fermentation, and performing bacterial cell disruption to obtain a crude enzyme preparation containing the glucose isomerase and the D-psicose 3-epimerase; and
(3) catalyzing glucose by using the crude enzyme preparation as a catalyst to perform isomerization to obtain D-psicose.

2. The method according to claim 1, wherein in Step (1), an amino acid sequence of the glucose isomerase is any one selected from the following:
(a) the sequence shown in SEQ ID NO: 1;
(b) an amino acid sequence that is obtained by substituting, deleting or adding one or more amino acids to/from the sequence shown in SEQ ID NO: 1 and has protein activity remaining unchanged; or
(c) an amino acid sequence that is at least 90% homologous with the sequence shown in SEQ ID NO: 1 and has protein activity same as or similar to that of the sequence shown in SEQ ID NO: 1; and
an amino acid sequence of the D-psicose 3-epimerase is any one selected from the following:
(A) the sequence shown in SEQ ID NO: 2;
(B) an amino acid sequence that is obtained by substituting, deleting or adding one or more amino acids to/from the sequence shown in SEQ ID NO: 2 and has protein activity remaining unchanged; or
(C) an amino acid sequence that is at least 90% homologous with the sequence shown in SEQ ID NO: 2 and has protein activity same as or similar to that of the sequence shown in SEQ ID NO: 2.

3. The method according to claim 2, wherein
the gene encoding the glucose isomerase is optimized according to the codon preference of the Bacillus subtilis, wherein a nucleotide sequence after the optimization is shown in SEQ ID NO: 3; and
the gene encoding the D-psicose 3-epimerase is optimized according to the codon preference of the Bacillus subtilis, wherein a nucleotide sequence after the optimization is shown in SEQ ID NO: 4.

4. The method according to claim 1, wherein the fermentation in Step (2) is specifically cultivation at 37° C. and 200 rpm for 48 h.

5. The method according to claim 1, wherein the fermentation medium in Step (2) comprises water and the following components: 10 g/L of peptone, 5 g/L of yeast extract, 2.5 g/L of monopotassium phosphate, 15 g/L of potassium hydrogen phosphate, 0.1 g/L of manganese chloride tetrahydrate, 0.1 g/L of magnesium sulfate heptahydrate, and 6 g/L of glucose.

6. The method according to claim 1, wherein before the engineered bacteria are inoculated into the fermentation medium for mixed fermentation, Step (2) further comprises the step of preparing a seed broth with the engineered bacteria: inoculating the engineered bacteria into an LB seed culture medium, and performing overnight cultivation at 37° C. and 200 rpm to obtain the seed broth.

7. The method according to claim 1, wherein the bacterial cell disruption in Step (2) is specifically: mixing lysozyme and a fermentation broth according to a proportion of a mass volume ratio being 0.1‰, and performing reactions at 37° C. and 200 rpm for 1 h.

8. The method according to claim 1, wherein a temperature of the isomerization in Step (3) is 60° C., and a time ranges from 2 h to 24 h.

9. The method according to claim 1, wherein after the isomerization, Step (3) further comprises sequentially filtering, purifying, chromatographically separating, and concentrating reaction products to separately obtain the D-psicose and fructose.

10. Composite engineered bacteria, comprising Bacillus subtilis expressing glucose isomerase derived from Thermus thermophilus and Bacillus subtilis expressing D-psicose 3-epimerase derived from Ruminococcus sp.

11. An application of the composite engineered bacteria according to claim 10 in producing D-psicose, or application thereof in preparing blood sugar and lipid lowering products.


SEQUENCE LISTING (18 sequences)

SEQ ID NO: 1 - protein, 387 aa, synthetic construct
MYEPKPEHRFTFGLWTVGNLGRDPFGDAVRERLDPVYVVHKLAELGAYGVNLHDEDLIPRGTPPQERDQIVRRFKKALEETGLKVPMVTANLFSDPAFKDGAFTSPDPWVRAYALRKSLETMDLGAELGAEIYVVWPGREGAEVEATGKARKVWDWVREALNFMAAYAEDQGYGYRFALEPKPNEPRGDIYFATVGSMLAFIHTLDRPERFGLNPEFAHETMAGLNFVHAVAQVLDAGKLFHIDLNDQRMSRFDQDLRFGSENLKAAFFLVDLLESSGYQGPRHFDAHALRTEDEEGVWAFARGCMRTYLILKERAEAFREDPEVKELLAAYYQQDPAALALLGPYSREKAEALKRAELPLEAKRRRGYGLERLDQLAVEYLLGVRG

SEQ ID NO: 2 - protein, 291 aa, synthetic construct
MKYGIYYAYWEKEWNGDYKYYIDKISKLGFDILEISCGAFSDYYTKDQELIDIGKYAKEKGVTLTAGYGPHLNESLSSSEPNTQKQAISFWKETLRKLKLMDIHMVGGALYGYWPVDYSKPFDKKRDLENSIKNMKIISQYAEEYDIMMGMEVLNRFEGYMLNTCDEALAYVEEVGSSNVGVMLDTFHMNIEEDNIAAAIRKAGDRLCHFHIGEGNRKVPGKGMLPWNEIGQALRDINYQHAAVMEPFVMQGGTVGHDIKIWRDIIGNCSEVTLDMDAQSALHFVKHVFEV

SEQ ID NO: 3 - DNA, 1164 bp, synthetic construct
  source 1..1164: mol_type = other DNA
atgtatgaaccaaaacccgagcacaggtttacgtttggcctgtggaccgttggtaatctgggccgtgatccatttggcgacgcggtgcgtgagcgtctggacccggtttatgtcgtgcacaaactggccgaactgggcgcctacggcgtgaacttgcatgatgaggaccttatcccgcgtggaaccccgcctcaggagcgggatcagattgtgcgccgtttcaagaaagctttagaagagactggtctgaaggttccgatggttaccgcaaatctgttcagcgatccggcgttcaaagatggtgcgttcacctcgccggatccgtgggtacgtgcatatgccttgaggaagtccctggagacgatggatttaggcgcggagctgggtgcggagatctacgtggtgtggccgggtcgcgagggcgctgaggttgaagcgaccggtaaggctcgtaaggtgtgggactgggttcgtgaagcgctcaacttcatggctgcgtatgcagaggaccagggctacggttaccgctttgcgttggagccgaagccgaatgaaccgagaggcgatatttactttgccacggtgggctccatgctggcgttcatccacaccctggacagacctgaacgctttggtctgaacccggaatttgctcatgaaaccatggcaggtttgaactttgttcatgcggtggcgcaggttctggacgccggtaaactgtttcatattgatctcaacgatcaacgtatgagccgtttcgaccaagacctgcgtttcggcagcgaaaacctgaaggcagcattcttcctggttgatttgctggagtctagcggttaccaaggtccgcgtcattttgacgcgcacgcgctgcgcaccgaagacgaagagggtgtttgggcgtttgcacgtggctgcatgcgtacctatctgatcctgaaagagcgtgcagaggcgttccgcgaggacccggaagtcaaagaactgttggccgcgtattatcagcaggatccggcagcgttggcgttgctgggtccatacagccgtgaaaaagcggaggcacttaagcgcgcggaactgccgctggaagctaaacgccgtcgtggttacggcctggagcgcctggaccaactggccgtcgaatatctgttaggggtgcgtggttaa

SEQ ID NO: 4 - DNA, 876 bp, synthetic construct
  source 1..876: mol_type = other DNA
atgaaatatggtatttattacgcttattgggaaaaggaatggaatggagattacaaatattatatagataaaattagcaaactgggctttgatattctggaaattagctgcggcgcgtttagcgattattataccaaagatcaggaactgattgatattggcaaatatgcgaaagaaaaaggcgtgaccctgaccgcgggctatggcccgcatctgaacgaaagcctgagcagcagcgaaccgaacacccagaaacaggcgattagcttttggaaagaaaccctgcgcaaactgaaactgatggatattcatatggtgggcggcgcgctgtatggctattggccggtggattatagcaaaccgtttgataaaaaacgcgatctggaaaacagcattaaaaacatgaaaattattagccagtatgcggaagaatatgatattatgatgggcatggaagtgctgaaccgctttgaaggctatatgctgaacacctgcgatgaagcgctggcgtatgtggaagaagtgggcagcagcaacgtgggcgtgatgctggatacctttcatatgaacattgaagaagataacattgcggcggcgattcgcaaagcgggcgatcgcctgtgccattttcatattggcgaaggcaaccgcaaagtgccgggcaaaggcatgctgccgtggaacgaaattggccaggcgctgcgcgatattaactatcagcatgcggcggtgatggaaccgtttgtgatgcagggcggcaccgtgggccatgatattaaaatttggcgcgatattattggcaactgctctgaagttacattagatatggacgctcaaagtgcgttgcactttgtaaaacatgtatttgaagtctaa

SEQ ID NO: 5 - DNA, 1164 bp, synthetic construct
  source 1..1164: mol_type = other DNA
atgtatgaaccgaaaccggaacatcgctttacctttggcctgtggaccgtgggcaacgtgggccgcgatccgtttggcgatgcggtgcgcgaacgcctggatccggtgtatgtggtgcataaactggcggaactgggcgcgtatggcgtgaacctgcatgatgaagatctgattccgcgcggcaccccgccgcaggaacgcgatcagattgtgcgccgctttaaaaaagcgctggaagaaaccggcctgaaagtgccgatggtgaccgcgaacctgtttagcgatccggcgtttaaagatggcgcgtttaccagcccggatccgtgggtgcgcgcgtatgcgctgcgcaaaagcctggaaaccatggatctgggcgcggaactgggcgcggaaatttatgtggtgtggccgggccgcgaaggcgcggaagtggaagcgaccggcaaagcgcgcaaagtgtgggattgggtgcgcggcgcgctgaactttatggcggcgtatgcggaagatcagggctatggctatcgctttgcgctggaaccgaaaccgaacgaaccgcgcggcgatatttattttgcgaccgtgggcagcatgctggcgtttattcataccctggatcgcccggaacgctttggcctgaacccggaatttgcgcatgaaaccatggcgggcctgaactttgtgcatgcggtggcgcaggtgctggatgcgggcaaactgtttcatattgatctgaacgatcagaaaatgagccgctttgatcaggatctgcgctttggcagcgaaaacctgaaagcggcgttttttctggtggatctgctggaaagcagcggctatcagggcccgcgccattttgatgcgcatgcgctgcgcaccgaagatgaagaaggcgtgtgggcgtttgcgcgcggctgcatgcgcacctatctgattctgaaagaacgcgcgaaagcgtttcgcgaagatccggaagtgaaagaaattctggcgacctattatgaagaagatccggaagcgattggcctgctgggcccgtatagccgcgaaaaagcggaagcgctgaaacgcgcggaactgccgctggaagcgaaacgccgccgcggctatgatctggaacgcctggatcagctggcggtggaatatctgctgggcgtgcgcggctaa

SEQ ID NO: 6 - DNA, 876 bp, synthetic construct
  source 1..876: mol_type = other DNA
atgaaatatggcatttattatgcgtattgggaaaaagaatggaccggcgattattttaaatatattgataaagtgaaagaactgggctttgatattctggaaattagctgcggcgcgtttaaaaaagattataccaccgatgaacagctgctgcgcattggcgaatatgcgaaagaaaaaggcattattctgaccgcgggctatggcccgcagaaagatgaaaacctggcgagcccggatgaagcggtgcagaaaaacgcgattgaattttttaaagaaaccctgcgcaaactgcatctgatgaacattcatattctgggcggcggcctgtatagctattggccggtggattttaacagcgatattaacaaagaacgcgattttgaaaccagcgtgaaaaacatgcgcctgattgcgaaatatgcggaagaatatgatattaccctgggcatggaagtgctgaaccgctttgaaggctatctgattaacacctgcgatgaatgcctgcgctatattgatgcggtgggcaacaaccgcgtgggcgtgatgctggatacctttcatatgaacattgaagaagataacattggcgatgcgattcgcaaagcgggcgataaactggtgcattttcatatgggcgaacgcaaccgcaaagtgccgggcaaaggcaccatgccgtgggcggaaattggccaggcgctgcgcgatattcattatcagcatgcggcggtgatggaaccgtttgtgctgcagggcggcaaagtgggccaggatattaaaatttggcgcaacctgctgccggataccagcgaagcgaaactggatgcggatgcgaaacatagctgcgaatttgtgcgccatgtgtttgaactgtaa

SEQ ID NO: 7 - DNA, 41 bp, synthetic construct
  source 1..41: mol_type = other DNA
ataaaaaaggagacatgaacgatgtatgaaccaaaacccga

SEQ ID NO: 8 - DNA, 45 bp, synthetic construct
  source 1..45: mol_type = other DNA
tcttggaattgtgctgaagctttaaccacgcacccctaacagata

SEQ ID NO: 9 - DNA, 21 bp, synthetic construct
  source 1..21: mol_type = other DNA
agcttcagcacaattccaaga

SEQ ID NO: 10 - DNA, 27 bp, synthetic construct
  source 1..27: mol_type = other DNA
cgttcatgtctccttttttatgtactg

SEQ ID NO: 11 - DNA, 52 bp, synthetic construct
  source 1..52: mol_type = other DNA
ataaaaaaggagacatgaacgatgaaatatggtatttattacgcttattggg

SEQ ID NO: 12 - DNA, 51 bp, synthetic construct
  source 1..51: mol_type = other DNA
tcttggaattgtgctgaagcttagactttcaaatacatgttttacaaagtg

SEQ ID NO: 13 - DNA, 21 bp, synthetic construct
  source 1..21: mol_type = other DNA
agcttcagcacaattccaaga

SEQ ID NO: 14 - DNA, 27 bp, synthetic construct
  source 1..27: mol_type = other DNA
cgttcatgtctccttttttatgtactg

SEQ ID NO: 15 - DNA, 20 bp, synthetic construct
  source 1..20: mol_type = other DNA
acagccattgaacatacggt

SEQ ID NO: 16 - DNA, 19 bp, synthetic construct
  source 1..19: mol_type = other DNA
ccttggtaaccgctagact

SEQ ID NO: 17 - DNA, 20 bp, synthetic construct
  source 1..20: mol_type = other DNA
acagccattgaacatacggt

SEQ ID NO: 18 - DNA, 26 bp, synthetic construct
  source 1..26: mol_type = other DNA
ttccatgcccatcataatatcatatt